(19) United States
(12) Patent Application Publication
(10) Pub. No.: US 2025/0143583 A1
(43) Pub. Date: May 8, 2025

(54) BODY TEMPERATURE MEASUREMENT DEVICE

(71) Applicant: Nippon Telegraph and Telephone Corporation, Tokyo (JP)

(72) Inventors: Kazuyoshi Ono, Tokyo (JP); Hiroyoshi Togo, Tokyo (JP)

(21) Appl. No.: 18/836,487

(22) PCT Filed: Apr. 6, 2022
(86) PCT No.: PCT/JP2022/017154

§ 371 Date: Aug. 7, 2024

(51) Int. Cl. A61B 5/01; A61B 5/00; G01K 1/14; G01K 7/04; G01K 13/20

(52) U.S. Cl. CPC A61B 5/01; A61B 5/6803; G01K 1/14; G01K 7/04; G01K 13/20; A61B 2562/0271; A61B 2562/04

(57) ABSTRACT

A body temperature measurement apparatus includes a holding mechanism, a first temperature sensor, a second temperature sensor, a third temperature sensor, a fourth temperature sensor, and a fifth temperature sensor, and a sixth temperature sensor. The first temperature sensor and the second temperature sensor are provided in the holding mechanism and measure a temperature of sides of a nose root portion of a measurement target person. The third temperature sensor is provided in the holding mechanism and measures a temperature of a nose top portion of the measurement target person. The fourth temperature sensor is provided in the holding mechanism and measures a temperature of a glabella of the measurement target person. The fifth temperature sensor and the sixth temperature sensor are provided in the holding mechanism and measure a temperature of the back sides of ear helices of the measurement target person.

### CROSS-REFERENCE TO RELATED APPLICATIONS

This application is a national phase entry of PCT Application No. PCT/JP2022/017154, filed on Apr. 6, 2022, which application is hereby incorporated herein by reference.

### TECHNICAL FIELD

The present invention relates to a body temperature measurement apparatus for measuring a temperature of a face of a measurement target person.

### BACKGROUND

It is known that when physical change occurs in a user due to emotional movements called emotions, autonomic nerve activity causes vasoconstriction and dilation, resulting in change in a blood flow. For example, when a person feels comfortable, an action of a sympathetic nerve weakens, while an action of a parasympathetic nerve becomes strong, which speeds up a blood flow in blood vessels and increases a blood flow. On the other hand, when the person feels uncomfortable, the action of the sympathetic nerve becomes strong, while the action of the parasympathetic nerve weakens, which slows down the blood flow in the blood vessels and decreases the blood flow.

When the blood flow increases, the skin temperature increases, and when the blood flow decreases, the skin temperature decreases. Particularly in a nose portion, since blood vessels pass through a small gap between skin and a nose bone, it is easy for a skin temperature to be affected by change in the blood flow. Therefore, it has been frequently reported that acquiring a nose temperature of a user is useful for estimation of emotions of the user. It is expected that a database for the estimation of the emotions of the user will be created by continuously measuring the nose temperature of the user on a daily basis (NPL 1, NPL 2, and NPL 3).

### CITATION LIST

### Non Patent Literature

NPL 1 Naoto Yoshida, Tomoko Yonezawa, “Robot-mediated communication through human's breath, heartbeat and temperature,” Human-Agent Interaction Symposium, P-27, pp. 216-221, 2015.
NPL 2 Yasushi Nagamine et al., “Evaluation of Emotions by Nasal Skin Temperature on Auditory Stimulus and Olfactory Stimulus,” Institute of Electrical Engineers of Japan Journal C (Electronics, Information, and Systems Division Journal), Vol. 124, No. 9, 2004.
NPL 3 Mikiko Kumamoto, “Study on Evaluation Method of Stress—nasal skin temperature and psychological state”, Sanyo Rongyo, Vol. 16, pp. 39-48, 2009.

### SUMMARY

### Technical Problem

However, most of the related technologies acquire a temperature of a nose portion without contact using thermography, and a technology for easily and continuously measuring a temperature of a face including a nose portion without interfering with daily life of a measurement target person has not been proposed.

Embodiments of the present invention have been made to solve the problem as described above, and an object of embodiments of the present invention is to make it possible to measure a temperature of a face including a nose portion easily and continuously without interfering with daily life of a measurement target person.

### Solution to Problem

A body temperature measurement apparatus according to embodiments of the present invention includes a holding mechanism mounted on a measurement target person; a first temperature sensor and a second temperature sensor provided in the holding mechanism and configured to measure a temperature of each of left and right sides of a nose root portion of the measurement target person; a third temperature sensor provided in the holding mechanism and configured to measure a temperature of a nose top portion of the measurement target person; a fourth temperature sensor provided in the holding mechanism and configured to measure a temperature of a glabella of the measurement target person; a fifth temperature sensor and a sixth temperature sensor provided in the holding mechanism and configured to measure temperatures of respective back sides of right and left ear helices of the measurement target person; a processing circuit configured to acquire measurement values from the first temperature sensor, the second temperature sensor, the third temperature sensor, the fourth temperature sensor, the fifth temperature sensor, and the sixth temperature sensor; a transmission circuit configured to transmit the measurement values acquired by the processing circuit to a set transmission destination; and a power supply circuit configured to supply a supply voltage to the first temperature sensor, the second temperature sensor, the third temperature sensor, the fourth temperature sensor, the fifth temperature sensor, the sixth temperature sensor, the processing circuit, and the transmission circuit.

### Advantageous Effects

As described above, according to embodiments of the present invention, since the first temperature sensor, the second temperature sensor, the third temperature sensor, the fourth temperature sensor, the fifth temperature sensor, and the sixth temperature sensor are provided in the holding mechanism worn by the measurement target person, it is possible to measure a temperature of a face including a nose portion easily and continuously without interfering with daily life of the measurement target person.

### BRIEF DESCRIPTION OF THE DRAWINGS

FIG. 1 is a perspective view illustrating a partial configuration of a body temperature measurement apparatus according to an embodiment of the present invention.

FIG. 2 is a configuration diagram illustrating a partial configuration of the body temperature measurement apparatus according to the embodiment of the present invention.

### DETAILED DESCRIPTION OF ILLUSTRATIVE EMBODIMENTS

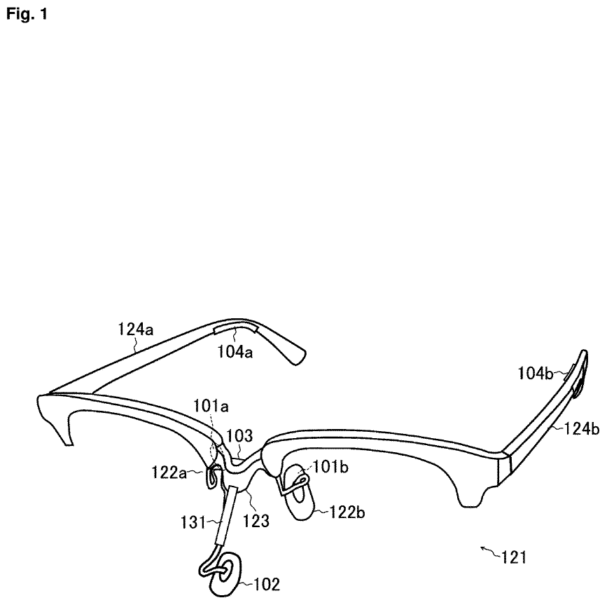

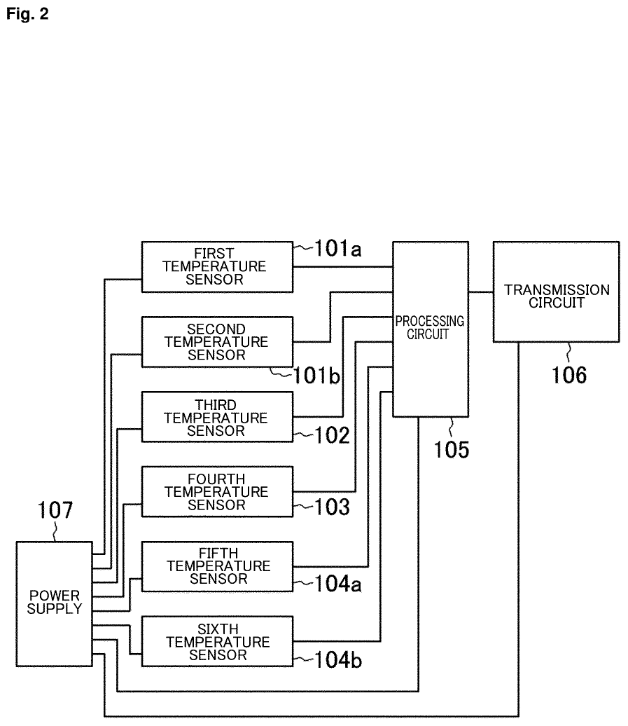

Hereinafter, a body temperature measurement apparatus according to an embodiment of the present invention will be described with reference to FIGS. 1 and 2. This body temperature measurement apparatus includes a holding mechanism 121, a first temperature sensor 101a, a second temperature sensor 101b, a third temperature sensor 102, a fourth temperature sensor 103, a fifth temperature sensor 104a, and a sixth temperature sensor 104b.

The holding mechanism 121 is worn by the measurement target person. The first temperature sensor 101a and the second temperature sensor 101b are provided in the holding mechanism 121 and measure a temperature (skin surface temperature) of the left and right sides of a nose root portion of the measurement target person. For example, the first temperature sensor 101a measures a temperature on the right side of the nose root portion of the measurement target person. Further, the second temperature sensor 101b measures a temperature on the left side of the nose root portion of the measurement target person.

The third temperature sensor 102 is provided in the holding mechanism 121 and measures a temperature (skin surface temperature) of a nose top portion of the measurement target person. The fourth temperature sensor 103 is provided in the holding mechanism 121 and measures a temperature (skin surface temperature) of a glabella of the measurement target person.

The fifth temperature sensor 104a and the sixth temperature sensor 104b are provided in the holding mechanism 121 and measure a temperature (skin surface temperature) of back sides of left and right ear helices of the measurement target person. For example, the fifth temperature sensor 104a measures the temperature of the back side of the right ear helix of the measurement target person. For example, the sixth temperature sensor 104b measures the temperature of the back side of the left ear helix of the measurement target person.

Further, this body temperature measurement apparatus includes a processing circuit 105, a transmission circuit 106, and a power supply circuit 107. The processing circuit 105 acquires measurement values from the first temperature sensor 101a, the second temperature sensor 101b, the third temperature sensor 102, the fourth temperature sensor 103, the fifth temperature sensor 104a, and the sixth temperature sensor 104b. The transmission circuit 106 transmits the measurement value acquired by the processing circuit 105 to a set transmission destination according to a set format. The transmission circuit 106 sets an external apparatus such as a smartphone, a tablet terminal, or a personal computer as the transmission destination and transmits the measurement value to the transmission destination through wireless communication such as Bluetooth (registered trademark) or wireless local area network (LAN), or wired communication.

The power supply circuit 107 supplies a supply voltage to the first temperature sensor 101a, the second temperature sensor 101b, the third temperature sensor 102, the fourth temperature sensor 103, the fifth temperature sensor 104a, the sixth temperature sensor 104b, the processing circuit 105, and the transmission circuit 106. The processing circuit 105, the transmission circuit 106, and the power supply circuit 107 can be built into the holding mechanism 121.

The processing circuit 105 and the transmission circuit 106 can be configured of computer equipment including a CPU, a main storage apparatus, an external storage apparatus, a network connection, and the like. Each function can be realized by the CPU operating according to a program expanded to the main storage apparatus.

For example, the holding mechanism 121 can be a glasses frame worn by the measurement target person. The first temperature sensor 101a and the second temperature sensor 101b are provided in respective pads 122a and 122b of the glasses frame, and measure a skin temperature of the measurement target person that the pads 122a and 122b face.

The third temperature sensor 102 is provided at a tip of a support rod 131 extending downward from a bridge 123 of the glasses frame, which is provided at the bridge 123, and measures a skin temperature of the measurement target person at the tip of the support rod 131. The fourth temperature sensor 103 is provided in the bridge 123 and measures a skin temperature of the measurement target person that the bridge 123 faces.

The fifth temperature sensor 104a and the sixth temperature sensor 104b are provided in respective moderns 124a and 124b of the glasses frame, and measure a skin temperature of the measurement target person that the moderns 124a and 124b face.

Further, the first temperature sensor 101a, the second temperature sensor 101b, the third temperature sensor 102, the fifth temperature sensor 104a, and the sixth temperature sensor 104b are used in contact with skin of the measurement target person, and can be configured of a thermocouple, and the fourth temperature sensor 103 can be configured of an infrared sensor (thermopile).

It goes without saying that individual temperature measurement results from the first temperature sensor 101a, the second temperature sensor 101b, the third temperature sensor 102, the fourth temperature sensor 103, the fifth temperature sensor 104a, and the sixth temperature sensor 104b are useful, but a combination of these makes it possible to acquire, for example, a signal regarding emotions or physiological states, such as change in an autonomic nerve controlling a left brain obtained by acquiring temperature changes at a left nose root portion and a nose top portion, and change in an autonomic nerve controlling a right brain obtained by acquiring temperature change at a right nose root portion and the nose top portion.

For example, it is possible to acquire (estimate) change in the autonomic nerve controlling the left brain on the basis of temperature change at the left nose root portion and the nose top portion. Further, it is possible to acquire (estimate) change in the autonomic nerve controlling the right brain on the basis of temperature change at the right nose root portion and the nose top portion. Further, it is possible to acquire (estimate) change in the autonomic nerve controlling the left and right centers on the basis of temperature change at the glabella and the nose top portion. It is possible to discern whether intuition or logical emotion is dominant from a left-right asymmetrical temperature change and a temperature gradient.

The holding mechanism 121 is not limited to the glasses frame, and for example, the holding mechanism 121 can be a face shield worn by the measurement target person. In this case, it is possible to acquire a daily continuous body temperature while the measurement target person wearing the face shield, which is effective in preventing infection.

Further, the holding mechanism 121 can be a goggle (for example, a VR goggle) that is worn by the measurement target person. In this case, continuous body temperature change can be fed back to virtual reality, making it possible to improve the sense of reality or sense of unity of virtual reality.

As described above, according to embodiments of the present invention, since the first temperature sensor, the second temperature sensor, the third temperature sensor, the fourth temperature sensor, the fifth temperature sensor, and the sixth temperature sensor are provided in the holding mechanism worn by the measurement target person, it is possible to measure a temperature of a face including a nose portion easily and continuously without interfering with daily life of the measurement target person.

Some or all of the embodiments are also described in the following supplements, but are not limited to the following.

### Supplement 1

A body temperature measurement apparatus, including:
a holding mechanism mounted on a measurement target person;
a first temperature sensor and a second temperature sensor provided in the holding mechanism and configured to measure a temperature of each of left and right sides of a nose root portion of the measurement target person;
a third temperature sensor provided in the holding mechanism and configured to measure a temperature of a nose top portion of the measurement target person;
a fourth temperature sensor provided in the holding mechanism and configured to measure a temperature of a glabella of the measurement target person;
a fifth temperature sensor and a sixth temperature sensor provided in the holding mechanism and configured to measure temperatures of respective back sides of right and left ear helices of the measurement target person;
a processing circuit configured to acquire measurement values from the first temperature sensor, the second temperature sensor, the third temperature sensor, the fourth temperature sensor, the fifth temperature sensor, and the sixth temperature sensor;
a transmission circuit configured to transmit the measurement values acquired by the processing circuit to a set transmission destination; and
a power supply circuit configured to supply a supply voltage to the first temperature sensor, the second temperature sensor, the third temperature sensor, the fourth temperature sensor, the fifth temperature sensor, the sixth temperature sensor, the processing circuit, and the transmission circuit.

### Supplement 2

The body temperature measurement apparatus according to supplement 1,
wherein the holding mechanism is a glasses frame worn by the measurement target person,
the first temperature sensor and the second temperature sensor are provided in both respective pads of the glasses frame, and measure a skin temperature of the measurement target person that the pads face,
the third temperature sensor is provided at a tip of a support rod extending downward from a bridge of the glasses frame, the support rod being provided at the bridge, and measures a skin temperature of the measurement target person at the tip of the support rod,
the fourth temperature sensor is provided in the bridge and measures a skin temperature of the measurement target person that the bridge faces, and
the fifth temperature sensor and the sixth temperature sensor are provided in both respective moderns of the glasses frame, and measure a skin temperature of the measurement target person that the moderns face.

### Supplement 3

The body temperature measurement apparatus according to supplement 1,
wherein the holding mechanism is a face shield worn by the measurement target person.

### Supplement 4

The body temperature measurement apparatus according to supplement 1,
wherein the holding mechanism is a goggle worn by the measurement target person. Supplement 5

The body temperature measurement apparatus according to any one of supplements 1 to 4,
wherein the processing circuit, the transmission circuit, and the power supply circuit are built into the holding mechanism.

Supplement 6

The body temperature measurement apparatus according to any one of supplements 1 to 5,
wherein the first temperature sensor, the second temperature sensor, the third temperature sensor, the fifth temperature sensor, and the sixth temperature sensor are configured of a thermocouple, and
the fourth temperature sensor is configured of an infrared sensor.

The present invention is not limited to the embodiments described above, and it will be obvious that many modifications and combinations can be implemented within the technical idea of the present invention by those skilled in the art.

### Reference Signs List

101a First temperature sensor
101b Second temperature sensor
102 Third temperature sensor
103 Fourth temperature sensor
104a Fifth temperature sensor
104b Sixth temperature sensor
105 Processing circuit
106 Transmission circuit
107 Power supply circuit
121 Holding mechanism
122a, 122b Pad
123 Bridge
124a, 124b Modern
131 Support rod

1-6. (canceled)

7. A body temperature measurement apparatus, comprising:
a holding mechanism configured to be mounted on a measurement target person;
a first temperature sensor in the holding mechanism and configured to measure a temperature of a left side of a nose root portion of the measurement target person;
a second temperature sensor in the holding mechanism and configured to measure a temperature of a right side of the nose root portion of the measurement target person;
a third temperature sensor in the holding mechanism and configured to measure a temperature of a nose top portion of the measurement target person;
a fourth temperature sensor in the holding mechanism and configured to measure a temperature of a glabella of the measurement target person;
a fifth temperature sensor in the holding mechanism and configured to measure a temperature of a back side of a right ear helix of the measurement target person;
a sixth temperature sensor in the holding mechanism and configured to measure a temperature of a back side of a left ear helix of the measurement target person;
a processing circuit configured to acquire measurement values from the first temperature sensor, the second temperature sensor, the third temperature sensor, the fourth temperature sensor, the fifth temperature sensor, and the sixth temperature sensor;
a transmission circuit configured to transmit the measurement values acquired by the processing circuit to a set transmission destination; and
a power supply circuit configured to supply a supply voltage to the first temperature sensor, the second temperature sensor, the third temperature sensor, the fourth temperature sensor, the fifth temperature sensor, the sixth temperature sensor, the processing circuit, and the transmission circuit.

8. The body temperature measurement apparatus according to claim 7,
wherein the holding mechanism is a glasses frame worn by the measurement target person.

9. The body temperature measurement apparatus according to claim 8,
wherein the first temperature sensor and the second temperature sensor are provided in respective pads of the glasses frame and are configured to measure a skin temperature of the measurement target person that the respective pads face.

10. The body temperature measurement apparatus according to claim 8,
wherein the third temperature sensor is provided at a tip of a support rod extending downward from a bridge of the glasses frame, the support rod being provided at the bridge, and the third temperature sensor being configured to measure a skin temperature of the measurement target person at the tip of the support rod.

11. The body temperature measurement apparatus according to claim 8,
wherein the fourth temperature sensor is provided in a bridge of the glasses frame and is configured to measure a skin temperature of the measurement target person that the bridge faces.

12. The body temperature measurement apparatus according to claim 8,
Wherein the fifth temperature sensor and the sixth temperature sensor are provided in respective moderns of the glasses frame, and are configured to measure a skin temperature of the measurement target person that the respective moderns face.

13. The body temperature measurement apparatus according to claim 7,
wherein the holding mechanism is a face shield configured to be worn by the measurement target person.

14. The body temperature measurement apparatus according to claim 7,
wherein the holding mechanism is a goggle configured to be worn by the measurement target person.

15. The body temperature measurement apparatus according to claim 7,
wherein the processing circuit, the transmission circuit, and the power supply circuit are built into the holding mechanism.

16. The body temperature measurement apparatus according claim 7,
wherein the first temperature sensor, the second temperature sensor, the third temperature sensor, the fifth temperature sensor, and the sixth temperature sensor are each a thermocouple, and
the fourth temperature sensor is an infrared sensor.

17. A body temperature measurement method, comprising:
mounting a holding mechanism on a measurement target person;
measuring, by a first temperature sensor in the holding mechanism, a temperature of a left side of a nose root portion of the measurement target person;
measuring, by a second temperature sensor in the holding mechanism, a temperature of a right side of the nose root portion of the measurement target person;
measuring, by a third temperature sensor in the holding mechanism, a temperature of a nose top portion of the measurement target person;
measuring, by a fourth temperature sensor in the holding mechanism, a temperature of a glabella of the measurement target person;
measuring, by a fifth temperature sensor in the holding mechanism, a temperature of a back side of a right ear helix of the measurement target person;
measuring, by a sixth temperature sensor in the holding mechanism, a temperature of a back side of a left ear helix of the measurement target person;
acquiring, by a processing circuit, measurement values from the first temperature sensor, the second temperature sensor, the third temperature sensor, the fourth temperature sensor, the fifth temperature sensor, and the sixth temperature sensor; and
transmitting, by a transmission circuit, the measurement values acquired by the processing circuit to a set transmission destination.

18. The body temperature measurement method according to claim 17, further comprising:
supplying, a power supply circuit configured, a supply voltage to the first temperature sensor, the second temperature sensor, the third temperature sensor, the fourth temperature sensor, the fifth temperature sensor, the sixth temperature sensor, the processing circuit, and the transmission circuit.

19. The body temperature measurement method according to claim 17,
wherein the holding mechanism is a glasses frame worn by the measurement target person.

20. The body temperature measurement method according to claim 19,
wherein the first temperature sensor and the second temperature sensor are provided in respective pads of the glasses frame and are configured to measure a skin temperature of the measurement target person that the respective pads face.

21. The body temperature measurement method according to claim 19,
wherein the third temperature sensor is provided at a tip of a support rod extending downward from a bridge of the glasses frame, the support rod being provided at the bridge, and the third temperature sensor being configured to measure a skin temperature of the measurement target person at the tip of the support rod.

22. The body temperature measurement method according to claim 19,
wherein the fourth temperature sensor is provided in a bridge of the glasses frame and is configured to measure a skin temperature of the measurement target person that the bridge faces.

23. The body temperature measurement method according to claim 19,
Wherein the fifth temperature sensor and the sixth temperature sensor are provided in respective moderns of the glasses frame, and are configured to measure a skin temperature of the measurement target person that the respective moderns face.

24. The body temperature measurement method according to claim 17,
wherein the holding mechanism is a face shield configured to be worn by the measurement target person.

25. The body temperature measurement method according to claim 17,
wherein the holding mechanism is a goggle configured to be worn by the measurement target person.

26. The body temperature measurement method according to claim 17,
wherein the processing circuit and the transmission circuit are built into the holding mechanism.
